(19) United States
(12) Patent Application Publication
(10) Pub. No.: US 2005/0191343 A1
(43) Pub. Date: Sep. 1, 2005

(54) Micellar systems useful for delivery of lipophilic or hydrophobic compounds

(75) Inventor: Likan Liang, Boyds, MD (US)

Correspondence Address: Heller Ehrman White & McAuliffe, Suite 300, 1666 K Street, N.W., Washington, DC, 20006, US

(73) Assignee: Shire Laboratories, Inc., Rockville, MD (US)

(21) Appl. No.: 10/995,942

(22) Filed: Nov. 24, 2004

Related U.S. Application Data
(60) Provisional application No. 60/566,157, filed on Apr. 28, 2004.
(60) Provisional application No. 60/541,389, filed on Feb. 2, 2004.
(60) Provisional application No. 60/525,572, filed on Nov. 26, 2003.

(51) Int. Cl. A61K 9/127

(52) U.S. Cl. 424/450

(57) ABSTRACT

The present invention is directed to reverse micellar formulations for the delivery of hydrophobic or lipophilic compounds, particularly therapeutic compounds.

### FIELD OF THE INVENTION

The present invention is directed to reverse micellar formulations for the delivery of hydrophobic or lipophilic compounds, particularly therapeutic compounds.

### BACKGROUND OF THE INVENTION

There are quite a few ways to improve the oral bioavailability of hydrophobic or lipophilic therapeutic compounds. Solubility enhancement, particle size reduction, permeability enhancement, p-glycoprotein inhibition as well as modified release are some of the most frequently used approaches.

For example, in oil-in-water microemulsion systems (o/w microemulsion), hydrophobic therapeutic compounds are normally solubilized in the oil phase as very small droplets, which are thermodynamically stabilized by surfactants. See U.S. Pat. No. 6,458,373. In self-emulsifying drug delivery systems (SEDDS), hydrophobic/lipophilic therapeutic compounds are dissolved in “oily” solvents and co-solvents, together with emulsifying agents/surfactants, which upon dilution in water or bodily fluid will form emulsions or similar structures (S. A. Charman, W. N. Charman, M. C. Rogge, T. D. Wilson, F. J. Dutko and C. W. Pouton, Self-emulsifying drug delivery systems: formulation and biopharmaceutic evaluation of an investigational lipophilic compound, Pharm. Res. 1992 Jan. 9(1):87-93).

Drug delivery systems may also include absorption enhancers to improve the oral bioavailability of hydrophobic therapeutic compounds. Amphiphilic molecules, having both hydrophilic and hydrophobic moieties in the same molecule, are surface-active agents (surfactants) and have been widely used as solubility enhancers and absorption enhancers. Upon contact with water, amphiphiles form various structures depending on such factors as their intrinsic properties, the ratio of water to amphiphiles and the presence of other components such as oils. At a high water-to-amphiphile ratio, micelles or emulsions may form, whereas at a low water-to-amphiphile ratio, the so-called L2 phase or reverse micelles or water-in-oil microemulsions may form. When mixed with an aqueous fluid such as water or body fluid to certain degree, reverse micelles may inverse, forming micelles, mixed micelles, emulsions, or other more complex structures or vesicles. Although formulations of reverse micelles have been shown to significantly improve the oral bioavailability of poorly absorbed water-soluble drugs (P. P. Constantinides, L. Liang, D. Fast, S. Dagar, L. He, L. Li, K. Opeifa, Bioavailability Enhancement of Leuprolide upon Intraduodenal Administration in Dogs from Lipid Polymer Micelles (LPM™), 2002 AAPS meeting, Toronto, Canada; and U.S. Pat. No. 6,429,200, Reverse Micelles for Delivery of Nucleic Acids), they have heretofore not been used extensively for the oral delivery of hydrophobic or lipophilic drugs.

There remains a need in the art for the efficient delivery and absorption of hydrophobic/lipophilic therapeutic compounds.

### SUMMARY OF THE INVENTION

The current invention provides formulations and methods for the delivery of biologically active hydrophobic and/or lipophilic therapeutic compounds to an animal. The present invention also discloses formulations and methods to improve the oral bioavailability of biologically active hydrophobic and/or lipophilic therapeutic compounds.

In particular, the current invention discloses formulations and methods to improve the solubility of biologically active hydrophobic and/or lipophilic therapeutic compounds, while also improving the oral absorption of said therapeutic compounds. The current invention further discloses methods to increase water-solubility of said therapeutic compounds.

The compositions are in the form of reverse micelles, which are comprised of one or more non-ioinic surfactants or a mixture of non-ionic and ionic surfactants, a hydrophilic phase composed of one or more hydrophilic solvents and/or solubilizers and/or aqueous media, and one or more therapeutically active, hydrophobic agents.

The compositions optionally further contain p-glycoprotein inhibitors, absorption enhancers or promoters, tight junction modulators, lipid membrane mobilizers, and antioxidants, as well as other typical pharmaceutically acceptable excipients such as buffering agents, flavorants, etc.

### DETAILED DESCRIPTION OF THE INVENTION

The formulations of the present invention are reverse micelle systems, which are composed of one or more surfactants, a continuous phase, a hydrophilic phase and one or more biologically active hydrophobic and/or lipophilic therapeutic compounds. As used herein, “reverse micelle” means “reverse micellar solution (L2)”, “reverse anisotropic nematic (N2)”, or “reverse micellar cubic (I2)” systems.

The reverse micelle formulations optionally contain solubilizers to increase the solubility of the biologically active hydrophobic and/or lipophilic therapeutic compounds in the formulations and/or in water or body fluids. Solubilizers can also provide a base for solubilizing the hydrophobic and/or lipophilic therapeutic compounds upon dilution by water or body fluid.

The reverse micelle systems comprising one or more surfactants, a continuous phase, a hydrophilic phase, one or more of said therapeutic compounds, and optionally one or more solubilizers, contain less than 15%, preferably less than 10%, more preferably less than 5%, and most preferably less than 2% triglycerides.

The reverse micelle systems can optionally include inhibitors known in the art, such as p-glycoprotein inhibitors (T. Chang, L. Z. Benet, M. F. Hebert, The effect of water-soluble vitamin E on cyclosporin pharmacokinetics in healthy volunteers, Clin. Pharmacol. Ther. 1996 Mar, 59(3):297-303), in order to improve the gastrointestinal absorption of the said therapeutic compounds.

The reverse micelle systems of the present invention may further contain other additives, such as absorption enhancers or promoters, tight junction modulators, lipid membrane mobilizers, antioxidants, preservatives, buffering agents, flavorants or any other pharmaceutically acceptable additives known in the art.

Surfactants

Generally, the surfactants included in the formulations of the present invention may be chosen from non-ionic surfactants, or combinations of non-ionic surfactants and ionic surfactants. Non-ionic surfactants include, but are not limited to, one or more fatty acid esters or their amide or ether analogues, or hydrophilic derivatives thereof, such as: monoesters or diesters, or hydrophilic derivatives thereof, or mixtures thereof; monoglycerides or diglycerides, or hydrophilic derivatives thereof, or mixtures thereof; mixtures having enriched mono- or/and diglycerides, or hydrophilic derivatives thereof; monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof; and fatty acid derivatives of amines, polyamines, polyimines, aminoalcohols, aminosugars, hydroxyalkylamines, hydroxypolyimines, peptides, polypeptides, or the ether analogues thereof. In this class, preferred are surfactants comprising, or enriched in, fatty acid moieties having 6-12 carbon atoms; more preferably having 6-8, 6-10, 6-12, 8-10 or 8-12 carbon atoms. The term “hydrophilic derivatives” as used herein means surfactants derivatized with hydrophilic components such that additional hydrophilic moieties are added to the surfactant molecules or to a partial structure of the surfactant molecules. Hydrophilic derivatives of surfactants also include partially derivatized surfactants, which are a mixture of the surfactant and its hydrophilic derivatives. As such, products of transesterification or other similar transformations of oils, alcohols and other surfactants with hydrophilic materials such as PEG, polypropylene glycol, saccharides, oligosaccharides, polysaccharides, and polyols, are included in the present invention.

Another class from which surfactants may be chosen is the ionic or Zwitterionic surfactants, such as fatty acid salts, bile salts, sulfates, sulfonates, sulfosuccinates, carboxylates, lactylates, phospholipids and derivatives, quaternary ammonium salts, amine salts, polyethoxylated ammonium salts, and mixtures thereof. Hydrophilic derivatives of such surfactants, such as PEG-phospholipids, are also included in the present invention.

The compositions of the present invention contain one or more non-ionic surfactants, or combinations of one or more non-ionic surfactants and one or more ionic surfactants where in the ratio of non-ionic surfactants to ionic surfactants is from about 99.99:0.01 to about 10:90. The HLB values (hydrophilic-lipophilic-balance) of the non-ionic surfactants are preferably >4, and more preferably have an HLB value of from about 5˜20.

The surfactants contain less than 15%, preferably less than 10%, more preferably less than 5%, and most preferably less than 2% by weight of triglycerides.

The amount of surfactants in the formulations of the present invention is between about 0.001 and about 99.8% by weight.

Examples of surfactants include, but are not limited to: medium chain transesterification products of oils and alcohols; monoglycerides or diglycerides or mixtures thereof; polyethylene glycol fatty acid monoesters or diesters or mixtures thereof; polyethylene glycol sorbitan fatty acid esters; polyethylene glycol alkyl ethers; propylene glycol fatty acid monoesters or diesters or mixtures thereof; POE-POP block copolymer fatty acid monoesters or diesters or mixtures thereof; sugar esters; bile salts; fatty acid salts; bisalkyl sulfosuccinate salts; phospholipids; hydrophilic derivatives of phospholipids; fatty acid derivatives of polyamines or polyimines or aminoalcohols or aminosugars or peptides or polypeptides; or mixtures of the above surfactants.

More specific examples of surfactants are: PEG-8 caprylic/capric glycerides (Labrasol, Acconon MC-8), PEG-6 caprylic/capric glycerides (Softgen 767, Acconon CC-6), PEG-12 caprylic /capric glycerides (Acconon CC-12), PEG-35 castor oil (Cremophor EL), PEG-40 castor oil (Cremophor RH), PEG-60 corn glycerides (Crovol M70,; lauroyl macrogol-32 glycerides (Gelucire 44/14), PEG-23 lauryl ether (Brij 35), PEG-8 laurate (MAPEG 400 ML), vitamin E TPGS, PEG-20 sorbitan monooleate (Tween 80), PEG-dipalmitoyl phosphatidylethanolamine, PEG-distearoyl phosphatidylethanolamine, bile acid and bile salts, CTAB, DODAB, and sodium bis(2-ethylhexyl) sulfosuccinate.

Continuous Phase

The continuous phase will comprise surfactants or solubilizers, or combinations of surfactants and solubilizers. Part or all of the therapeutic compound(s) is/are dissolved in the continuous phase.

Other components in the reverse micelle systems of the present invention may or may not be solubilized in the continuous phase. When no other solubilizers are used, the bulk of the surfactant(s) functions as the continuous phase as well as the solubilizer. The amount of the continuous phase comprises 50-99.9% by weight of the formulation.

Solubilizers (Optional):

The surfactants can function as solubilizers in which the therapeutic compound(s) is/are solubilized. In addition to the said surfactants, however, one or more of the following materials can be added to the formulation as solubilizers:

Amphiphilic compounds: such as fatty acid esters, ethers or amides of alcohols, aminoalcohols, glycols, polyols, saccharides or oligosaccharides or polysaccharides, oxyalkylene oligomers or polymers or block polymers, amines, polyimines, hydroxyalkylamines, hydroxypolyimines, peptides, polypeptides, or hydrophilic derivatives thereof; and hydrophilic derivatives of fatty acids, polyglycerized fatty acids.

Ionic or Zwitterionic surfactants: such as fatty acid salts, bile salts, sulfates, sulfonates, carboxylates, lactylates, phospholipids and derivatives thereof, and quaternary ammonium salts.

Complexing agents: such as charge-complex agents (for example, fatty acids, organic acids and chelating agents); and inclusion complexing agents (for example, cyclodextrins and derivatives).

Solvents/co-solvents: such as hydrophobic or hydrophilic solvents/co-solvents.

Esters, ethers, alcohols, fatty alcohols, aromatic alcohols, polyols, oxyalkylene oligomers or polymers or block polymers, amines, amides, fatty acids, or water.

Or mixtures of the above solubilizers may be used.

The solubilizers should contain less than 15%, preferably less than 10%, more preferably less than 5%, and most preferably less than 2% by weight of C6-C25 fatty acid triglycerides.

The amount of solubilizer(s) in the formulations of the present invention is 0˜99.8% by weight.

Some examples of such solubilizers include, but are not limited to: fatty acid monoesters or diesters or mixtures thereof of glycols such as ethylene glycols or propylene glycols or butylenes glycols; monoglycerides or diglycerides or mixtures thereof; polyglycerized fatty acids, polyethylene glycol fatty acid monoesters or diesters or mixtures thereof; POE-POP block copolymer fatty acid monoesters or diesters or mixtures thereof; polyethylene glycol sorbitan fatty acid esters; sorbitan fatty acid esters; ethylene glycol or diethylene glycol or triethylene glycol or polyethylene glycol alkyl ethers; phospholipids and derivatives thereof; PEG-phospholipids; PEGs; alcohols; fatty alcohols; fatty acids; and mixtures of the foregoing solubilizers.

Some more specific examples of solubilizers include: propylene glycol dicaprylate/dicaprate (Captex 200), propylene glycol monocaprylate (Capmul PG-8), propylene glycol caprylate/caprate (Labrafac PG), propylene glycol dicaprylate (Captex 100), propylene glycol diethylhexanoate, propylene glycol monolaurate (Capmul PG-12), glyceryl caprylate/caprate (Capmul MCM), glyceryl monocaprylate (Capmul MCMC-8, Imwitor 308), glyceryl monooleate (Capmul GMO), capric acid monoglyceride (Imwitor 312), PEG-6 corn oil (Labrafil M 2125), sorbitan monooleate (Span 80);

sodium lauryl sulfate, sodium taurocholate, lecithin, lyso-lecithin, phosphatidyl glycerol, polyethylene glycol-phosphatidyl ethanolamines, cetyl trimethyl ammonium bromide, lauryl betaine;

oleic acid, caprylic acid, capric acid;

citric acid, tartaric acid, ascorbic acid, EDTA;

cyclodextrin (various forms and derivatives thereof); and

acetyl triethylcitrate, triethylcitrate, ethyl oleate, ethyl caprylate, triacetin; tetrahydrofurfuryl alcohol PEG ether (glycofurol), m-PEG, diethylene glycol monoethyl ether (Transcutol), diethylene glycol monobutyl ether, ethylene glycol monoethyl ether; ethanol, isopropanol, benzyl alcohol, ethylene glycol, propylene glycol, glycerol, sorbitol, mannitol, polyvinylalcohol, cellulose derivatives; polyethylene glycol (PEG 400 etc.), polypropylene glycol, POE-POP block polymers; pyrrolidones, N-alkylpyrrolidones, N-hydroxyalkylperrolidones, N-methylpyrrolidone, piperidones, N-alkylpiperidones, polyvinylpyrrolidones.

Inhibitors

Optionally, the formulations of the present invention can also include inhibitors, such as enzyme inhibitors, and P-glycoprotein inhibitors. The concentration of these inhibitors is in accordance with the knowledge in the art.

Other Additives

The formulations may also contain other additives known in the art, such as: absorbable osmotic gradient agents, such as glucose or sucrose; buffering agents; antioxidants; preservatives, or other suitable pharmaceutically acceptable additives; known absorption promoters or enhancers; tight junction modulators, such as palmitoyl carnitine, and lipid membrane mobilizers, such as cholesterol or surfactants or lipids that are incorporated into the cellular lipid membrane of intestinal epithelia and act to lower the surface tension of the membrane allowing for easier transcellular passage of lipophilic molecules.

Hydrophilic Phase

The hydrophilic phase in the formulations of the present invention contains one or more hydrophilic solvents and/or solubilizers and/or aqueous media. Water may or may not be present in the hydrophilic phase. The hydrophilic phase comprises from about 0.1 to about 50% by weight of the formulations. Other components may be present in the hydrophilic phase, such as solubilizers, water-miscible solvents, water-soluble surfactants, ionic surfactants, complexing agents, and other additives.

Therapeutic Compounds

The terms “therapeutic compound” or “drug” or “(pharmaceutically) active agent” are used in the present specification and claims to mean any compound useful for therapeutic, nutritional, or diagnostic purposes. Further, the term encompasses one or more of such compounds, or one or more of such compounds in composition with any other (non-hydrophobic) active agent(s). Additionally, the present invention is contemplated as useful for the delivery of such agents to any animal, but preferably mammals, and most preferably humans.

The reverse micelle systems of the present invention are applicable to the oral or mucosal delivery of any hydrophobic or lipophilic therapeutic compounds. In the present formulations, there may be more than one such hydrophobic drug, or such a drug in combination with any other agent, hydrophobic or not.

The present invention is not limited to only certain active agents, but is for example applicable to any poorly water-soluble compound for which controlled release delivery is desired. Non-limiting examples of such active agents would include albendazole, albuterol, acyclovir, adriamycin, carbamazepine, oxcarbazepine, amiodarone, amlodipine, amphetamine, amphotericin B, atorvastatin, atovaquone, azithromycin, baclofen, bicalutamide, busulfan, butenafine, calcipotriene, calcitriol, camptothecin, capsaicin, carotenes, celecoxib, cerivastatin, chlorpheniramine, cimetidine, ciprofloxacin, cisapride, cetirizine, clarithromycin, clemastine, codeine, cyclosporin, danazol, dantrolene, dexchlorpheniramine, digoxin, dirithromycin, donepezil, efavirenz, eprosartan and other sartans, etodolac, etoposide, famotidine, fentanyl, finasteride, fluconazole, flurbiprofen, fluvastatin, fosphenytoin, frovatriptan, gabapentin, gemfibrozil, glibenclamide, glyburide, glimepiride, griseofulvin, halofantrine, ibuprofen, irinotecan, isotretinoin, itraconazole, ivermectin, ketoconazole, ketorolac, lamotrigine, angiotensin converting enzyme (ACE) or NEP inhibitors, fenofibrate or fibric acid derivatives, fexofenadine, flutamide, glipizide, glyburide, isradipine, loratadine, lovastatin, melphalan, nifedipine, leflunomide, loperamide, lycopenes, mifepristone, mefloquine, methadone, methoxsalen, metronidazole, miconazole, midazolam, miglitol, mitoxantrone, nabumetone, nalbuphine, naratriptan, nelfinavir, nilutamide, nizatidine, oxaprozin, paclitaxel, pentazocine, pioglitazone, pizotefin, pravastatin, probucol, pyridostigmine, raloxifene, rofecoxib, repaglinide, rifapentine, rimexolone, rizatriptan, rosiglitazone, saquinavir, sibutramine, sildenafil citrate, simvastatin, sirolimus, spironolactone, sumatriptan, tacrine, tacrolimus, tamoxifen, tamsulosin, targretin, tazarotene, teniposide, terbinafine, tiagabine, tizanidine, topiramate, topotecan, toremifene, tramadol, tretinoin, troglitazone, trovafloxacin, verteporfin, vigabatrin, vitamin A, vitamin D, vitamin E, vitamin K, zafirlukast, zileuton, zolmitriptan, zolpidem, zopiclone, proton pump inhibitors such as lansoprazole, esomeprazole, omeprazole, and rabeprazole, MAP kinase inhibitors, ICE inhibitors such as pralnacasan, pseudoephedrine, indomethacin, naproxen, estrogens, testosterones, steroids, phenytoin, ergotamines and cannabinoids, pharmaceutically acceptable salts, isomers, prodrugs (e.g. esters) and derivatives thereof, and mixtures thereof.

Preferred hydrophobic actives include albuterol, acyclovir, adriamycin, carbamazepine, oxcarbazepine, topiramate, eprosartan, cyclosporin, griseofulvin, angiotensin converting enzyme (ACE) or NEP inhibitors, fenofibrate, fexofenadine, flutamide, glipizide, glyburide, isradipine, loratadine, lovastatin, melphalan, nifedipine, proton pump inhibitors such as lansoprazole, esomeprazole, omeprazole, and rabeprazole, MAP kinase inhibitors, pralnacasan, pseudoephedrine, indomethacin, naproxen, estrogens, testosterones, steroids, phenytoin, sumatriptan, ergotamines or cannabinoids, or pharmaceutically acceptable salts, isomers, or prodrugs or derivatives thereof. More preferred are those selected from carbamazepine, oxcarbazepine, eprosartan, fenofibrate or fibric acid derivatives, fexofenadine, glipizide, topiramate, cyclosporin, lansoprazole, esomeprazole and rabeprozole, or pharmaceutically acceptable salts, isomers, or prodrugs or derivatives thereof. Most preferred in the reverse micelle compositions of the present invention are therapeutic compounds chosen from fenofibrate or fibric acid derivatives, carbamazepine, topiramate, eprosartan, and cyclosporin.

The concentration of drug in the formulations depends, of course, on the desired dosage of the active agent. It will be appreciated that the amount of a compound of the invention required for use in treatment will vary not only with the particular compound selected but also the nature of the condition for which treatment is required, and the desired dosage regimen, it being understood that extended or sustained release dosage forms such as those of the instant invention are usually intended to reduce the number of dosages taken per day or to sustain a desired plasma level. Additionally, the necessity or desire for other components of the dosage core will serve to dictate the maximum percentage of drug. In general, however, the core of a dosage unit according to the present invention will contain anywhere from about 0.5% by weight to about 90% by weight of the drug, preferably from about 1 to about 50%, and more preferably from about 1 to about 10%.

Reverse Micelle Formulations

In one embodiment, the reverse micelle systems of the present invention comprise one or more surfactants, a continuous phase, a hydrophilic phase and one or more of said therapeutic compounds. In accordance with this embodiment, the continuous phase comprises the bulk of said surfactants, which are selected from non-ionic surfactants or combinations of non-ionic surfactants and ionic surfactants. Further, the reverse micelle systems comprise less than 15%, preferably less than 10%, more preferably less than 5%, and most preferably less than 2% by weight of triglycerides. The hydrophilic phase comprises one or more hydrophilic solvents, solubilizers or aqueous media, or combinations thereof. The substantial amount of the therapeutic compound(s) is/are solubilized in the continuous phase.

In another embodiment, the reverse micelle systems comprise one or more fatty acid esters or ethers or hydrophilic derivatives thereof, a continuous phase, a hydrophilic phase and one or more of said therapeutic compounds. In accordance with this embodiment, the continuous phase comprises the bulk of said esters or ethers or hydrophilic derivatives thereof. Further, the reverse micelle systems comprise less than 15%, preferably less than 10%, more preferably less than 5%, and most preferably less than 2% by weight of triglycerides.

In yet another embodiment, the reverse micelle systems comprise one or more surfactants, one or more solubilizers, a continuous phase, a hydrophilic phase and one or more of said therapeutic compounds. In accordance with this embodiment, the reverse micelle systems comprise less than 15%, preferably less than 10%, more preferably less than 5%, and most preferably less than 2% by weight of triglycerides. The solubilizers can be miscible in the continuous phase, or in the hydrophilic phase, or in both phases. Further, the systems can contain more than one solubilizer, in which some of the solubilizers may be miscible in the continuous phase (or the hydrophobic/lipophilic phase), increasing the solubility of the hydrophobic/lipophilic therapeutic compounds in the formulations, while other solubilizers may be miscible in the hydrophilic phase, increasing the water-solubility of the said therapeutic compounds upon mixing with the body fluid. In accordance with this embodiment, the reverse micelle systems comprise less than 15%, preferably less than 10%, more preferably less than 5%, and most preferably less than 2% by weight of triglycerides.

In yet another embodiment, the reverse micelle systems comprise one or more surfactants, one or more solubilizers, a continuous phase, a hydrophilic phase and one or more of the said therapeutic compounds. In accordance to this embodiment, the solubilizers contain at least one complexing agent, which will form complexes with the therapeutic compounds and increase the water-solubility of the therapeutic compounds. In a preferred embodiment, the complexing agent is a cyclodextrin. Cyclodextrins may form inclusion complexes with said therapeutic compounds. In another, preferred embodiment, the complexing agent is an acid such as citric acid or oleic acid. In this embodiment, the acid may form a charge-complex with therapeutic compounds bearing 1°, 2° and 3° amine groups.

In still another embodiment, the reverse micelle systems comprise one or more surfactants, one or more solubilizers, a continuous phase, a hydrophilic phase, one or more inhibitors and one or more of said therapeutic compounds. In accordance with this embodiment, the inhibitors are selected from those known to one skilled in the art, such as p-glycoprotein inhibitors, which will improve the absorption of the therapeutic compounds.

In a further embodiment, the reverse micelle systems contain other additives, such as absorption enhancers or promoters, tight junction modulators, lipid membrane mobilizers, antioxidants, preservatives, buffering agents, flavorants or other pharmaceutically suitable additives known in the art.

In yet another embodiment, the reverse micelle systems comprise non-ionic surfactants or combinations of non-ionic surfactants and ionic surfactants wherein the non-ionic surfactants have HLB values greater than 4. Preferably, the HLB value of the non-ionic surfactants is between 5 and 20, and more preferably between 10 and 20.

Additionally, any of the systems of the present invention may include one or more water soluble solubilizers or additives, such as cyclodextrin, citric acid, glucose, sucrose, ionic surfactants, buffering agents, etc. (which are otherwise not soluble in many surfactants or solubilizers and are not suitable for use in most self-emulsifying drug delivery systems) to increase the water solubility of the therapeutic compounds and increase the absorption of the said therapeutic compounds in the gastrointestinal tract. The systems can also provide amphiphilic solubilizers for increased solubility of the said therapeutic compounds. In other words, the systems described herein can significantly improve the bioavailability of orally or mucosally administered therapeutic agents.

The reverse micelle systems can further contain other pharmaceutically acceptable excipients to form a gel, a semi-solid, a solid dispersion, such that the reverse micelle systems are absorbed in the solid form of the said excipients. The systems are compatible with many encapsulation materials such as gelatin or HPMC. The reverse micelle systems can be encapsulated by micro-encapsulation techniques known in the art, or in capsules (hard or soft gelatin capsules or capsules made of other materials such as starch), or in enterically coated capsules, or in coated capsules for controlled release, as powders, or in cachets, or made into tablets or liquid dosage forms.

Administration and Treatment

The present invention further provides a method of administering a dosage form containing the reverse micelles of the present invention to an animal, preferably a human. It is primarily contemplated that the dosage forms described herein are administered by an oral route. The desired dose may conveniently be -presented in-a single dose-or as divided dose administered at appropriate intervals, for example as two, three, four or more doses per day.

The formulations of the present invention are used to treat an abnormal condition, provide nutritional supplementation, and/or deliver diagnostic agents to a mammal, preferably human, in need thereof. Basically, the method of treating such a condition involves orally administering a dosage form containing the reverse micelle formulations of the present invention to the subject in need of treatment. The terms “treat”, “treating” and “treatment” are intended to include prevention of a condition or illness as well.

As preferred embodiments, formulations of the present invention that contain fenofibrate, carbamazepine, or topiramate as an active ingredient are used to treat hypertensive (fenofibrate) or epileptic (carbamazepine, topiramate) conditions in patients in a manner known in the art.

It will be appreciated by those skilled in the art that the active ingredients can be used in the form of pharmaceutically acceptable salts, free bases, prodrugs (e.g. esters) or derivatives and, in the case of chirally active ingredients, one can use one or both optical isomers, geometric isomers and mixtures thereof including racemic mixtures.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention now will be described in particularity with the following illustrative examples; however, the scope of the present invention is not intended to be, and shall not be, limited to the exemplified embodiments below.

### EXAMPLES

### Example 1

### Fenofibrate Reverse Micelle Systems

Appropriate amounts of surfactants and hydrophilic phase, as listed below, in Table 1, were vortex mixed briefly until uniformly dispersed. To the resulting reverse micelles, an appropriate amount of fenofibrate was added and vortex mixed, warming the mixture if fenofibrate is not readily solubilized. A transparent liquid was formed.

TABLE 1

| Ingredient | PD0106-89-11 | PD0106-79-1 |
|---|---|---|
| PEG-8 caprylic/capric | 9.5 | 9 |
| glycerides (Labrasol) | | |
| PEG 400 | — | 1 |
| Water | 0.5 | — |
| Fenofibrate | 0.54* | 0.6 |

Note:
Amounts in grams, unless otherwise indicated.
*Determined by HPLC in solubility study.

### Example 2

### Fenofibrate Reverse Micelle Systems Containing Hydrophilic Solubilizers

For formulations in which hydrophilic solubilizers were used, as listed below in Table 2, the hydrophilic solubilizers were premixed with the other components in the hydrophilic phase before mixing with the rest of the components. A transparent liquid was formed.

TABLE 2

| Ingredient | PD0106-89-12 | PD0106-79-2 | PD0106-79-3 |
|---|---|---|---|
| PEG-8 caprylic/capric | 9.5 | 9 | 9 |
| glycerides | | | |
| (Labrasol) | | | |
| SLS | 0.01 | 0.1 | — |
| Cyclodextrin | 0.025 | — | — |
| Diethylene glycol | — | — | 0.5 |
| monoethyl ether | | | |
| (Transcutol) | | | |
| PEG 400 | — | 0.7 | 0.5 |
| Water | 0.465 | 0.2 | — |
| Fenofibrate | 0.55* | 0.6 | 0.6 |

Note:
Amounts in grams, unless otherwise indicated.
*Determined by HPLC in solubility study.

### Example 3

### Fenofibrate Reverse Micelle Systems Containing Surfactant-Miscible Solubilizers

For formulations in which surfactant-miscible solubilizers were used, as listed below in Table 3, the surfactant-miscible solubilizers were premixed with the therapeutic compounds before mixing with the other components, warming the mixture if fenofibrate is not readily solubilized. A transparent liquid was formed.

TABLE 3

| Ingredient | 44^{#} | 41^{#} | 53^{#} | 56^{#} | 59^{#} | 38^{#} | 62^{#} | 65^{#} |
|---|---|---|---|---|---|---|---|---|
| PEG-8 caprylic/capric | 4.85 | 6.5 | — | 6 | 6 | 4.9 | 6 | 6 |
| glycerides | | | | | | | | |
| (Labrasol) | | | | | | | | |
| PEG-35 castor oil | — | — | 3.9 | — | — | — | — | — |
| (Cremophor EL) | | | | | | | | |
| Propylene glycol | — | — | — | — | — | 2.4 | 2.2 | 2.2 |
| dicaprylate/dicaprate | | | | | | | | |
| (Captex 200) | | | | | | | | |
| Propylene glycol | 4.85 | — | | — | — | 2.4 | — | — |
| monocaprylate | | | | | | | | |
| (Capmul PG-8) | | | | | | | | |
| PEG-4 lauryl ether | — | — | — | 3.7 | — | — | 1.5 | — |
| (Brij 30) | | | | | | | | |
| Glyceryl | — | 3.2 | — | — | — | — | — | — |
| caprylate/caprate | | | | | | | | |
| (Capmul MCM) | | | | | | | | |
| PEG-6 corn oil | — | — | 5.8 | — | — | — | — | — |
| (Labrafil M2125 CS) | | | | | | | | |
| PG-20 corn glycerides | — | — | — | — | 3.7 | — | — | 1.5 |
| (Crovol M40) | | | | | | | | |
| Water | 0.3 | 0.3 | 0.3 | 0.3 | 0.3 | 0.3 | 0.3 | 0.3 |
| Fenofibrate | 1 | 1 | 1 | 1 | 1 | 1 | 1 | 1 |

Note:
Amounts in grams, unless otherwise indicated.
^{#}Formulation series number PD0106-85

### Example 4

### Fenofibrate Reverse Micelle Systems Containing Both Hydrophilic and Surfactant-Miscible Solubilizers

For formulations in which both hydrophilic and surfactant-miscible solubilizers were used, as listed in Tables 4, 5 and 6, the surfactant-miscible solubilizers were premixed with the therapeutic compounds and the hydrophilic solubilizers were premixed with the other components in the hydrophilic phase before mixing with the rest of the components. A transparent liquid was formed.

TABLE 4

| Ingredient | 57^{#} | 58^{#} | 60^{#} | 61^{#} | 63^{#} | 64^{#} | 66^{#} | 67^{#} |
|---|---|---|---|---|---|---|---|---|
| PEG-8 | 6 | 6 | 6 | 6 | 6 | 6 | 6 | 6 |
| caprylic/ | | | | | | | | |
| capric | | | | | | | | |
| glycerides | | | | | | | | |
| (Labrasol) | | | | | | | | |
| Propylene | — | — | — | — | 2.2 | 2.2 | 2.2 | 2.2 |
| glycol | | | | | | | | |
| dicaprylate/ | | | | | | | | |
| dicaprate | | | | | | | | |
| (Captex 200) | | | | | | | | |
| PEG-4 lauryl | 3.7 | 3.7 | | | 1.5 | 1.5 | — | — |
| ether | | | | | | | | |
| (Brij 30) | | | | | | | | |
| PEG-20 corn | — | — | 3.7 | 3.7 | — | — | 1.5 | 1.5 |
| glycerides | | | | | | | | |
| (Crovol M40) | | | | | | | | |
| SLS | — | 0.005 | — | 0.005 | — | 0.005 | — | 0.005 |
| PEG 400 | 0.15 | — | 0.15 | — | 0.15 | — | 0.15 | — |
| Water | 0.15 | 0.29 | 0.15 | 0.29 | 0.15 | 0.29 | 0.15 | 0.29 |
| Fenofibrate | 1 | 1 | 1 | 1 | 1 | 1 | 1 | 1 |

Note:
Amounts in grams, unless otherwise indicated.
^{#}Formulation series number PD0106-85

TABLE 5

| | | | | | | | PD0-106- | |
|---|---|---|---|---|---|---|---|---|
| Ingredient | 35^{#} | 39^{#} | 42^{#} | 43^{#} | 45^{#} | 46^{#} | 89-14 | 40^{#} |
| PEG-8 | 6.9 | 4.9 | 6.5 | 6.5 | 4.85 | 4.85 | 4.7 | 4.85 |
| caprylic/ | | | | | | | | |
| capric | | | | | | | | |
| glycerides | | | | | | | | |
| (Labrasol) | | | | | | | | |
| Propylene | 2.65 | 2.4 | — | — | — | — | 2.4 | 2.43 |
| glycol | | | | | | | | |
| dicaprylate/ | | | | | | | | |
| dicaprate | | | | | | | | |
| (Captex 200) | | | | | | | | |
| Propylene | — | 2.4 | — | — | 4.85 | 4.85 | 2.4 | 2.43 |
| glycol | | | | | | | | |
| monocaprylate | | | | | | | | |
| (Capmul PG8) | | | | | | | | |
| Propylene | — | 2.4 | — | — | 4.85 | 4.85 | 2.4 | 2.43 |
| glycol | | | | | | | | |
| monocaprylate | | | | | | | | |
| (Capmul PG8) | | | | | | | | |
| Glyceryl | — | — | 3.2 | 3.2 | — | — | — | — |
| caprylate/ | | | | | | | | |
| caprate | | | | | | | | |
| (Capmul MCM) | | | | | | | | |
| PEG-6 corn | — | — | — | — | — | — | — | — |
| oil (Labrafil | | | | | | | | |
| M2125 CS) | | | | | | | | |
| Cyclodextrin | — | — | — | — | — | — | 0.05 | — |
| SLS | 0.05 | — | — | 0.005 | — | 0.005 | 0.01 | 0.005 |
| PEG 400 | 0.25 | 0.15 | 0.15 | — | 0.15 | — | — | — |
| Water | 0.15 | 0.15 | 0.15 | 0.29 | 0.15 | 0.29 | 0.44 | 0.29 |
| Fenofibrate | 1 | 1 | 1 | 1 | 1 | 1 | 0.76* | 1 |

Note:
Amounts in grams, unless otherwise indicated.
^{#}Formulation series number PD0106-85
*Determined by HPLC in solubility study.

TABLE 6

| | PD0106-115 | PD0106-115 | PD0106-115 |
|---|---|---|---|
| Ingredient | #75 | #59 | #65 |
| Glyceryl caprylate/ | 8.33 | 9.78 | 7.06 |
| caprate (Capmul MCM) | | | |
| Glyceryl monooleate | — | — | 1.11 |
| (Capmul GMO) | | | |
| PEG-35 castor oil | 9.78 | 9.78 | 5.56 |
| (Cremophor EL) | | | |
| Benzyl alcohol | 0.89 | — | 0.139 |
| SLS | 0.011 | 0.011 | 0.044 |
| Water | 0.56 | 0.56 | 0.78 |
| Fenofibrate | 1 | 1 | 1 |

Note:
Amounts in grams, unless otherwise indicated.

### Example 5

### Fenofibrate Transport Study in a Caco-2 Cell Model

Reverse micelles undergo inversion upon dilution by water or body fluid. Thus, a Labrasol solution (10%) containing fenofibrate is a mimic of the inversed reverse micelle systems. The effect of the reverse micelle systems on the transport of fenofibrate was measured against control and expressed as % enhancement, as listed in Table 7. For comparison purposes, some of the solubilizers are also included in Table 7.

In this example, Caco-2 cells were grown to confluence on permeable supports mounted in a chamber that has an apical side (AP) and a basolateral (BL) side. The fenofibrate-containing reverse micelles were added to the apical chamber to give a concentration of 0.2 mg/mL. Permeability coefficients can be determined as previously reported by Yazdania et.el (Yazdanian M, Glynn, S I, Wright J L, et al. 1998. Correlating partitioning and Caco-2 permeability of structurally diverse small molecular weight compounds. Pharm Res 15:1490-1494). Briefly, fenofibrate in Labrasol solutions were prepared at a known final concentration. For AP to BL experiments, the solution was placed on the apical side of the cells and samples were taken from basolateral side. The samples are analyzed by an HPLC. Transport rates (J) are determined by plotting cumulative amounts of drug permeated as a function of time. Alternatively, related enhancement ratio is used. The results show that the reverse micelle formulations are order of magnitude higher than solubilizer solutions of fenofibrate in the CaCo-2 transport model, suggesting much-improved bioavailability in vivo.

Table 7 shows the calculated related enhancement ratio from the Caco-2 transport study.

TABLE 7

| | | Diluted reverse | | |
|---|---|---|---|---|
| | Control | micelle (10% | Solubilizers | |
| Systems | (HBSS) | Labrasol) | Capmul PG-8 | Capmul MCM |
| Related | 1 | 6779 | 2.8 | 1.7 |
| enhancement | | | | |
| ratio | | | | |

### Example 6

### Stability of Fenofibrate Reverse Micelle Systems

Formulation PD01 06-92 was placed into gelatin capsules (LiCaps, CAPSUGEL) for a stability study according to the ICH guidelines. All other samples were placed in a stability chamber at 25° C. without humidity control. No crystal growth or phase separation was observed in any of the samples, as summarized in Table 8. Capsules were intact.

TABLE 8
Stability of Fenofibrate Reverse Micelles

| | | Initial | 1 month | 2 month | 3 month |
|---|---|---|---|---|---|
| Appearance | 25° C./ | No crystal | No crystal | No crystal | No crystal |
| | 60% RH | | | | |
| | 40° C./ | No crystal | No crystal | No crystal | No crystal |
| | 75% RH | | | | |
| Average | 25° C./ | 100.0 | 98.6 | 99.8 | 98.8 |
| Content (%) | 60% RH | | | | |
| | 40° C./ | — | 100.2 | 99.5 | 98.7 |
| | 75% RH | | | | |

### Example 7

### Reverse Micelle Systems Containing Carbamazepine

For carbamazepine formulations in which both hydrophilic and surfactant-miscible solubilizers were used, as listed in Table 9, the surfactant-miscible solubilizers were premixed with the therapeutic compounds and the hydrophilic solubilizers were premixed with the other components in the hydrophilic phase before mixing with the rest of the components. A transparent liquid was formed.

TABLE 9

| Ingredient | % w/w | Amt Prepared (g) |
|---|---|---|
| Water | 2.59 | 0.259 |
| SLS | 0.05 | 0.005 |
| Captex 200 P | 22.09 | 2.209 |
| Capmul PG8 | 22.09 | 2.209 |
| Labrasol | 44.09 | 4.409 |
| Carbamazepine | 9.09 | 0.909 |
| Total | 100 | 10 |

### Example 8

### Reverse Micelle Systems Containing Topiramate

For topiramate formulations in which both hydrophilic and surfactant-miscible solubilizers were used, as listed in Table 10, the surfactant-miscible solubilizers were premixed with the therapeutic compounds and the hydrophilic solubilizers were premixed with the other components in the hydrophilic phase before mixing with the rest of the components. A transparent liquid was formed.

TABLE 10

| Ingredient | % w/w | Amt Prepared (g) |
|---|---|---|
| Water | 2.59 | 0.259 |
| SLS | 0.05 | 0.005 |
| Captex 200 P | 22.09 | 2.209 |
| Capmul PG8 | 22.09 | 2.209 |
| Labrasol | 44.09 | 4.409 |
| Topiramate | 9.09 | 0.909 |
| Total | 100 | 10 |

### Example 9

### Reverse Micelle Systems for Water-Insoluble Drugs for Bioavailability Improvement and Food Effect Reduction

Reverse micelles and self-emulsifying drug delivery systems enhanced the oral bioavailability of fenofibrate up to 446% in dogs under fasted conditions compared to a commercially available product. Food effect was greatly reduced or even eliminated.

In this Example, stable reverse micelles (RM) along with stable self-emulsifying drug delivery systems (SEDDS) were developed as platform technologies for oral/mucosal delivery of water-insoluble drugs.

Reverse micelle formulations of carbamazepine and topiramate were made according to Examples 7 and 8. Reverse micelle formulations of fenofibrate and self-emulsifying formulations of fenofibrate were prepared in accordance with Tables 11 and 12. The formulations were filled in size 00 hard gelatin capsules. Stability of filled capsules was studied according to ICH guidelines.

TABLE 11

| Ingredient | RM (PD0106-121) | % w/w |
|---|---|---|
| Fenofibrate | 6.1 | 4.90 |
| Capmul MCM | 50.1 | 40.25 |
| Cremophor EL | 59.4 | 47.73 |
| Benzyl alcohol | 5.4 | 4.34 |
| Water | 3.4 | 2.73 |
| SLS | 0.0625 | 0.05 |

Amount in grams

TABLE 12

| Ingredient | SEDDS (PD0106-122) | % w/w |
|---|---|---|
| Fenofibrate | 8.5 | 6.79 |
| Capmul MCM | 76.1 | 60.83 |
| Labrasol | 11.3 | 9.03 |
| Benzyl alcohol | 8.5 | 6.79 |
| Span 80 | 11.3 | 9.03 |
| Ethanol | 9.4 | 7.51 |

Amount in grams

A crossover pharmacokinetic study of fenofibrate reverse micelle and self-emulsifying formulations in canine was carried out using a commercial product TriCor® tablet as the control. All dogs (n=6) received a dose of each test formulation in capsules and TriCor® tablet (all at 54 mg fenofibrate dose) under fed and fasted conditions with a 7-day washout period. Following each dose, PK samples were drawn at pre-dose, 0.25, 0.5, 1.0, 1.5, 2, 3, 4, 6, 9, 15 and 24 hr time points. Samples were analyzed for fenofibric acid using a validated LC/UV method.

Reverse micelle formulations of fenofibrate, carbamazepine and topiramate and self-emulsifying formulations of fenofibrate are stable at room temperature. A three-month stability study (25° C./60% RH and 40° C./75% RH) showed that RM and SEDDS formulations are stable and compatible with gelatin capsules. No fenofibrate crystals were observed in a 1:20 mixture of reverse micelle formulation with DI water or simulated intestinal fluid for up to 2 days, indicating that fenofibrate remains solubilized in the mixture (transparent) even after the inversion of the reverse micelles.

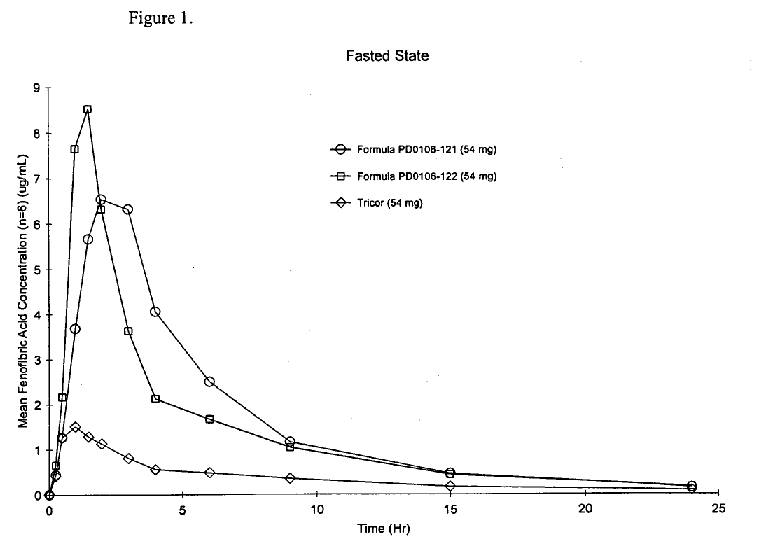

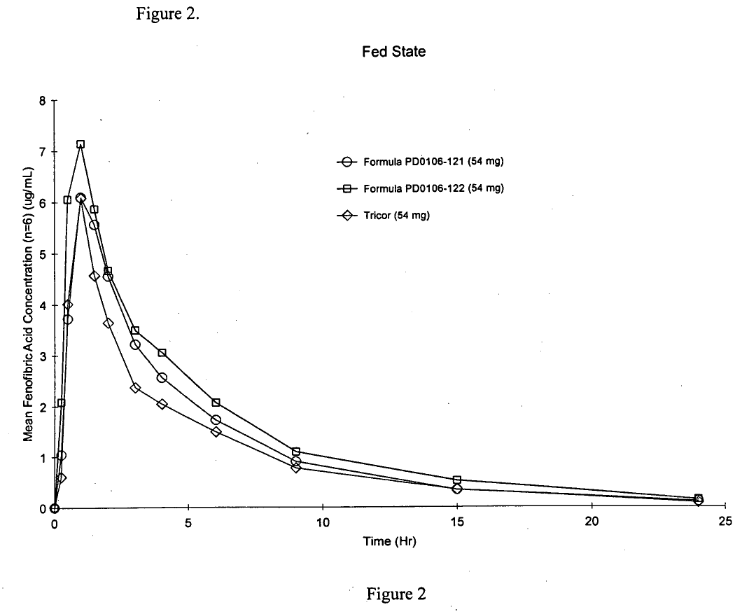

Results from this crossover pharamacokinetic study of reverse micelle and self-emulsifying formulations in canine are summarized in Table 13 and FIGS. 1 and 2. In the dog study, AUCs from the reverse micelles (PD0106-121), self-emulsifying formulation (PDO106-122) and TriCor® groups are 37.9, 33.1 and 8.5 μg *hr/mL under fasted conditions and 29.2, 35.9 and 25.2 μg *hr/mL under fed conditions, respectively. Under fasted conditions, AUCs from the reverse micelle and self-emulsifying groups are significantly higher (446% and 389%) than those from the TriCor® group. Changes in AUC between fed and fasted conditions are much smaller in the reverse micelle and self-emulsifying groups than in the TriCor® group, representing significant reduction of food effect and improvement of oral bioavailability.

In conclusion, reverse micelle formulations are shown to significantly reduce food effect and improve oral bioavailability of fenofibrate in the fasted state in dogs compared to the TriCor® control.

TABLE 13

| | RM | | SEDDS | | TriCor ® | |
|---|---|---|---|---|---|---|
| | Fasted | Fed | Fasted | Fed | Fasted | Fed |
| Tmax (hr) | 2.2 | 1.2 | 1.3 | 0.9 | 0.8 | 0.9 |
| Cmax (μg/mL) | 7.77 | 6.9 | 9.05 | 7.82 | 1.8 | 6.16 |
| AUCₗₐₛₜ | 37.9 | 29.2 | 33.1 | 35.9 | 8.5 | 25.2 |
| (hr*μg/mL) | | | | | | |
| Relative | 150 | 116 | 131 | 141 | 36 | 100 |
| Bioavail- | | | | | | |
| ability (%) | | | | | | |

1. A composition in the form of a reverse micelle, comprising a continuous phase containing one or more surfactants, a hydrophilic phase, and one or more biologically active hydrophobic therapeutic agents, wherein said one or more surfactants are selected from non-ionic surfactants, or combinations of non-ionic and ionic surfactants.

2. The composition of claim 1, which contains less than 15% by weight of triglycerides.

3. The composition of claim 1, which contains less than 2% by weight of triglycerides.

4. The composition of claim 1, wherein the one or more surfactants is/are selected from non-ionic surfactants.

5. The composition of claim 4, wherein the non-ionic surfactants are those with an HLB value of more than 4.

6. The composition of claim 4, wherein said one or more surfactants is/are fatty acid esters or their amide or ether analogues, or hydrophilic derivatives thereof, selected from:
monoesters or diesters, or hydrophilic derivatives thereof, or mixtures thereof;
monoglycerides or diglycerides, or hydrophilic derivatives thereof, or mixtures thereof;
mixtures having enriched mono- or/and diglycerides, or hydrophilic derivatives thereof, monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof;
and fatty acid derivatives of amines, polyamines, polyimines, aminoalcohols, aminosugars, hydroxyalkylamines, hydroxypolyimines, peptides, polypeptides, or the ether analogues thereof.

7. The composition of claim 6, wherein the one or more surfactants is/are selected from PEG-8 caprylic/capric glycerides (Labrasol, Acconon MC-8), PEG-6 caprylic/capric glycerides (Softgen 767, Acconon CC-6), PEG-12 caprylic /capric glycerides (Acconon CC-12), PEG-35 castor oil (Cremophor EL), PEG-40 castor oil (Cremophor RH), PEG-60 corn glycerides (Crovol M70, lauroyl macrogol-32 glycerides (Gelucire 44/14), PEG-23 lauryl ether (Brij 35), PEG-8 laurate (MAPEG 400 ML), vitamin E TPGS, PEG-20 sorbitan monooleate (Tween 80).

8. The composition of claim 6, wherein the one or more surfactants is/are selected from fatty acid moieties having 6-12 carbon atoms.

9. The composition of claim 1, wherein the surfactants comprise a combination of non-ionic and ionic surfactants.

10. The composition of claim 9, wherein the ratio of non-ionic to ionic surfactants is from about 99.99:0.01 to about 10:90.

11. The composition of claim 9, wherein the ionic surfactants are selected from PEG-dipalmitoyl phosphatidylethanolamine, PEG-distearoyl phosphatidylethanolamine, bile acid and bile salts, CTAB, DODAB, and sodium bis(2-ethylhexyl) sulfosuccinate.

12. The composition of claim 1, which further comprises one or more solubilizers.

13. The composition of claim 12, wherein said solubilizers are selected from amphiphilic compounds, ionic or Zwitterionic surfactants, complexing agents, solvents/co-solvents, or mixtures thereof.

14. The composition of claim 13, wherein said solubilizers are selected from propylene glycol dicaprylate/dicaprate (Captex 200), propylene glycol monocaprylate (Capmul PG-8), propylene glycol caprylate/caprate (Labrafac PG), propylene glycol dicaprylate (Captex 100), propylene glycol diethylhexanoate, propylene glycol monolaurate (Capmul PG-12), glyceryl caprylate/caprate (Capmul MCM), glyceryl monocaprylate (Capmul MCMC-8, lmwitor 308), glyceryl monooleate (Capmul GMO), capric acid monoglyceride (Imwitor 312), PEG-6 corn oil (Labrafil M 2125), oleic acid, caprylic acid, capric acid, acetyl triethylcitrate, triethylcitrate, ethyl oleate, ethyl caprylate, triacetin; tetrahydrofurfuryl alcohol PEG ether (glycofurol), diethylene glycol monoethyl ether (Transcutol), diethylene glycol monobutyl ether, ethylene glycol monoethyl ether; benzyl alcohol, polyvinylalcohol, POE-POP block polymers, pyrrolidones, N-alkylpyrrolidones, N-hydroxyalkylperrolidones, N-methylpyrrolidone, piperidones, N-alkylpiperidones, polyvinylpyrrolidones, sodium lauryl sulfate, sodium taurocholate, lecithin, lyso-lecithin, phosphatidyl glycerol, polyethylene glycol-phosphatidyl ethanolamines, cetyl trimethyl ammonium bromide, lauryl betaine, or mixtures thereof.

15. The composition of claim 12, wherein the solubilizers are present in an amount of 0 to about 99.8% by weight.

16. The composition of claim 1, wherein the hydrophilic phase comprises from about 0.1 to about 50% by weight of the composition.

17. The composition of claim 12, wherein the solubilizer(s) contain at least one complexing agent that will form complexes with therapeutic compounds.

18. The composition of claim 17, wherein the complexing agent is selected from cyclodextrin, citric acid or oleic acid.

19. The composition of claim 1, which further comprises one or more absorption enhancers, tight junction modulators, and/or lipid membrane mobilizers.

20. The composition of claim 1, which further comprises one or more P-glycoprotein inhibitors.

21. The composition of claim 1, wherein the therapeutic agents are one or more selected from: albendazole, albuterol, acyclovir, adriamycin, carbamazepine, oxcarbazepine, amiodarone, amlodipine, amphetamine, amphotericin B, atorvastatin, atovaquone, azithromycin, baclofen, bicalutamide, busulfan, butenafine, calcipotriene, calcitriol, camptothecin, capsaicin, carotenes, celecoxib, cerivastatin, chlorpheniramine, cimetidine, ciprofloxacin, cisapride, cetirizine, clarithromycin, clemastine, codeine, cyclosporin, danazol, dantrolene, dexchlorpheniramine, digoxin, dirithromycin, donepezil, efavirenz, eprosartan, ergotamine, etodolac, etoposide, famotidine, fentanyl, finasteride, fluconazole, flurbiprofen, fluvastatin, fosphenytoin, frovatriptan, gabapentin, gemfibrozil, glibenclamide, glyburide, glimepiride, griseofulvin, halofantrine, ibuprofen, irinotecan, isotretinoin, itraconazole, ivermectin, ketoconazole, ketorolac, lamotrigine, angiotensin converting enzyme (ACE) or NEP inhibitors, fenofibrate or fibric acid derivatives, fexofenadine, flutamide, glipizide, glyburide, isradipine, loratadine, lovastatin, melphalan, nifedipine, leflunomide, loperamide, lycopenes, mifepristone, mefloquine, methadone, methoxsalen, metronidazole, miconazole, midazolam, miglitol, mitoxantrone, nabumetone, nalbuphine, naratriptan, nelfinavir, nilutamide, nizatidine, oxaprozin, paclitaxel, pentazocine, pioglitazone, pizotefin, pravastatin, probucol, pyridostigmine, raloxifene, rofecoxib, repaglinide, rifapentine, rimexolone, rizatriptan, rosiglitazone, saquinavir, sibutramine, sildenafil citrate, simvastatin, sirolimus, spironolactone, sumatriptan, tacrine, tacrolimus, tamoxifen, tamsulosin, targretin, tazarotene, teniposide, terbinafine, tiagabine, tizanidine, topiramate, topotecan, toremifene, tramadol, tretinoin, troglitazone, trovafloxacin, verteporfin, vigabatrin, vitamin A, vitamin D, vitamin E, vitamin K, zafirlukast, zileuton, zolmitriptan, zolpidem, zopiclone, proton pump inhibitors such as lansoprazole, esomeprazole, omeprazole, and rabeprazole, MAP kinase inhibitors, ICE inhibitors, pseudoephedrine, indomethacin, naproxen, estrogens, testosterones, steroids, phenytoin, ergotamines and cannabinoids, pharmaceutically acceptable salts, isomers, prodrugs, and derivatives thereof.

22. The composition of claim 21, wherein the therapeutic agents are one or more selected from: albuterol, acyclovir, adriamycin, carbamazepine, oxcarbazepine, cyclosporin, eprosartan, griseofulvin, angiotensin converting enzyme (ACE) or NEP inhibitors, fenofibrate or fibric acid derivatives, fexofenadine, flutamide, glipizide, glyburide, isradipine, loratadine, lovastatin, melphalan, nifedipine, proton pump inhibitors, MAP kinase inhibitors, pralnacasan, pseudoephedrine, indomethacin, topiramate, naproxen, estrogens, testosterones, steroids, phenytoin, sumatriptan, ergotamines or cannabinoids, or pharmaceutically acceptable salts, isomers, or prodrugs or derivatives thereof.

23. The composition of claim 22, wherein the therapeutic agents are one or more selected from: carbamazepine, oxcarbazepine, eprosartan, fenofibrate or fibric acid derivatives, fexofenadine, glipizide, topiramate, cyclosporin, lansoprazole, esomeprazole and rabeprozole, or pharmaceutically acceptable salts, isomers, or prodrugs or derivatives thereof.

24. The composition of claim 23, wherein the therapeutic agents are one or more selected from: fenofibrate or fibric acid derivatives, carbamazepine, topiramate, eprosartan, and cyclosporin.

25. The composition of claim 1, wherein the reverse micelles are encapsulated by microencapsulation techniques, or in capsules (hard or soft gelatin capsules or capsules made of other materials such as starch), or in enterically coated capsules, or in coated capsules for controlled release, as powders, or in cachets, or made into tablets or liquid dosage forms.

26. A method for treating a condition or illness in a subject in need thereof, comprising orally administering a composition according to claim 1.
